(19) United States
(12) Patent Application Publication
(10) Pub. No.: US 2009/0221806 A1
(43) Pub. Date: Sep. 3, 2009

(54) Method for Producing a Beta-1,3-Glucan With Improved Characteristics

(76) Inventors: Werner Frohnwieser, Hilgertshausen (DE); Michael Volland, Karlsfeld (DE); Evi Wittmann, Traunreut (DE); Fabienne Skorupinsui, Carentan (FR); Jean Jacques Lebehot, Saint Pellerin (FR); Yves Lemoigne, Lieusaint (FR); Thomas Lötzbeyer, Eching (DE)

Correspondence Address: FULBRIGHT & JAWORSKI, LLP, 666 FIFTH AVE, NEW YORK, NY, 10103-3198, US

(21) Appl. No.: 10/544,820

(22) PCT Filed: Mar. 4, 2004
(86) PCT No.: PCT/EP04/02203

§ 371 Date: Sep. 10, 2007

(30) Foreign Application Priority Data
Mar. 4, 2003 (DE) 103 09 281.1

(51) Int. Cl. C07G 3/00; C12P 19/44

(52) U.S. Cl. 536/4.1; 435/74

(57) ABSTRACT

In this process for preparing a β-1,3-glucan, the glucan-containing matrix is treated with a protein having β(1,3)-glucanase activity, wherein the concentration of the glucanase amounts from 0.001 to 3.0% by weight. The glucan-containing matrix can be a fermentation broth, a culture medium or a suspension, where appropriate containing unsolved solids, cell constituents and/or cell fragments, or else a mycelium, a hydrocolloid or a powder preparation having a solvent proportion of from 20 to 99.9% by weight. The duration of the enzymatic treatment should be between 15 minutes and 24 hours and the treatment should be carried out continuously. The invention also envisages the glucan-containing matrix being filtered or centrifuged, after it has been treated enzymatically, and the glucan finally being separated off. A β-1,3-glucan which has been prepared in this way and which exhibits, for example, improved solubility in cold water, reduced proportions of insoluble constituents, an increased viscosity or improved filterability, is also claimed. A solid formulation and the use of these glucans for cosmetic applications, foodstuffs or oil production are also coclaimed.

The present invention relates to a process for preparing a β-1,3-glucan, to specific β-1,3-glucans, to a solid formulation and to the use of the specifically prepared β-1,3-glucans.

β-1,3-Glucans, which also include the scleroglucans, inter alia, are glucose molecules which are correspondingly linked to form polysaccharides.

Scleroglucans are water-soluble, nonionic natural polymers which are produced by a large number of filamentous fungi such as Sclerotium rolfsii. On an industrial scale, scleroglucans are obtained using aerobic, submerged cultures of selected strains. Sceroglucans consist of β-1,3-D-glucose molecules and have β-1,6-D-glucose side chains on every third sugar molecule. The average molecular weight is >10⁶ Da.

When used as an industrial polymer, scleroglucan is principally employed for thickening drilling mud in connection with oil production. However, it is just as customary to use it in adhesives, water-based paints, printing inks, cosmetics and in the pharmaceutical industry. In water, this biopolymer forms pseudoplastic solutions having shear-thinning properties and, furthermore, the biopolymer tolerates high temperatures and broad pH ranges and is also resistant to electrolytes.

Most scleroglucans are prepared economically by precipitating them from fermentation broth and isolating them as a solid. Because of their viscosity properties, it is generally not possible to separate off all the solids which are released during the fermentation prior to the precipitation step, which means that the dried and solid scleroglucans normally have certain proportions of water-insoluble solids in the form of cell fragments. These solids in turn remain unsolved when the scleroglucans are dissolved in water, something which results in the scleroglucans having to be subjected to additional purification in the case of applications which require the polysaccharides to have defined degrees of purity. To achieve this, the fermentation broths are first of all diluted, because of their elevated viscosity, and then, a filtration aid is added in preparation for the filtration step which follows. This procedure is very time- and energy-consuming, and the yield of purified polysaccharide of <50% is relatively low. The turbidity, which is in no way satisfactory, of the resulting scleroglucan solution is a further disadvantage in this connection.

A large number of processes, such as the process in accordance with U.S. Pat. No. 4,165,257, which describes the addition of a caustic enzyme, such as esperase, for degrading protein-like cell fragments, have been developed for circumventing these disadvantages. U.S. Pat. No. 4,119,491 also discloses the addition of solid, silicaceous materials which clarify the polysaccharides without any substantial loss of viscosity. DE-A 195 47 748 describes the addition of a detergent to the fermentation broth, with this resulting in phase separation and concentrating the polysaccharides in the top phase.

EP-A 514 890 proposes a mechanical process for purifying polysaccharide-containing solutions: in this process, a stirring device is used to mix an aqueous solution of the polysaccharides with a hydrophilic organic solvent, with this not, however, dissolving the polysaccharide.

Documents DE-A 3835771, U.S. Pat. No. 4,299,825 and U.S. Pat. No. 3,355,447 in each case describe processes for improving the filterability of polysaccharide-containing solutions by means of heat treatment, filtration or ultrafiltration. EP-B 049 012 also proposes an ultrafiltration, but in combination with an enzymatic treatment, for obtaining a concentrated solution of xanthan.

EP-B 039 962 recommends using of a Pellicularia sp.-derived enzyme complex having cell-lytic β(1,3)-glucanase and protease activities for degrading water-insoluble constituents in aqueous polysaccharide-containing solutions derived from fermenting Xanthomonas.

U.S. Pat. No. 4,416,990 protects an enzymatic process for clarifying impure xanthan gum, which at least contains bacterial cell constituents or microgels, by adding a polysaccharase preparation of Basidomycetes polyporaceae cellulase. DE-A 3 139 249 describes an enzymatic clarification of a natural xanthan resin in aqueous phase: in this case, a Basidomycetes sp. cellulase is used to remove bacterial cell residues or microgels.

All the above-described innovations in each case have a specific improvement as their goal, with the individual improvements essentially being directed towards two main properties of polysaccharide solutions:

On the one hand, the clarity of corresponding solutions should be improved and, on the other hand, filtration should be facilitated and the filtration result should be improved.

A large number of processes which are used to treat glucan-containing cell constituents with β-1,3-glucanases or with enzymes of equivalent activity have also been disclosed.

Thus, EP-B 440 725 discloses the preparation of a glucan from Saccharomyces cerevisiae, wherein an endo-β-glucanase in the form of laminarinase is used. U.S. Pat. No. 6,090,615 describes a process which uses β-1,3-glucanases to prepare a β-glucan-containing extract from a mycelium-containing culture medium. However, in this process, the glucanase is not employed on its own but, instead, in combination with chitinase and cellulase such that, with the mycelium being used as the starting material, the constituents contained in the mycelium are released by means of pulping. U.S. Pat. Nos. 5,250,436 and 4,810,646 have in each case previously described processes for obtaining glucan by degradation of glucan-containing matrices using laminarinase. In these processes, the binding structure of the glucans present in yeast cells is altered by means of an alkali treatment and a subsequent acid treatment, resulting in the glucans displaying viscosity properties which are typical depending on the yeast strain employed.

Recovery of microcapsules is given special emphasis in U.S. Pat. No. 5,521,089. In this process, yeast cells are treated with a β-1,3-glucanase, resulting in microcapsules which are suitable for enclosing hydrophobic liquids.

According to U.S. Pat. No. 6,284,509, modification of β-glucans, such as curdlan or laminarin, is achieved by using β-1,3-glucanases.

Taken overall, it is striking that the results achieved using the above-described processes do not lead simultaneously to improved rheological properties, enhanced solubilities and increased filtration yields.

The above-described disadvantages of the prior art have given rise to the object of the present invention, i.e. to provide a process for preparing a β-1,3-glucan, which process is used to obtain glucans which, if possible, exhibit an improved solubility in cold water, an increased viscosity and reduced turbidity as well as markedly reduced proportions of insoluble constituents and which is associated with markedly improved filterability.

This object was achieved by means of a corresponding process in which a glucan-containing matrix is treated with a protein possessing β(1,3)-glucanase activity.

It has been found, surprisingly, that, by using this process, it is possible on the basis of enzymatic activities, to release, into aqueous solutions, the glucan molecules which are bound to the mycelium in insoluble form. In accordance with the object, success is also achieved, when using this process, in increasing the viscosity of glucan-containing solutions and, in addition, in reducing the proportions of insoluble constituents as well as markedly improving the solubility of the glucans in cold water.

Matrices whose β-1,3-glucans have β(1,6)-glucose side chains have proved to be particularly suitable within the meaning of the present invention.

A process in which the protein employed is a β(1,3)-glucanase and, in particular, a protein which, in addition to the β(1,3)-glucanase activity, also exhibits a β(1,4)-glucanase activity, can also be regarded as being a preferred variant. The β(1,3)-glucanases which are preferably used by the present invention are produced by a variety of microorganisms, such as Trichoderma or Bacillus.

It has proved to be advisable, in connection with the present invention, if the concentration of the protein possessing β(1,3)-glucanase activity is between 0.001 and 3.0% by weight, in particular from 0.01 to 1.0% by weight, and particularly preferably from 0.1 to 0.5% by weight, in each case based on the reaction mixture.

Depending on the protein or enzyme selected and/or on its concentration, the present invention envisages reaction temperatures which are between 15 and 60° C., and preferably between 20 and 40° C., with room temperature having to be regarded as being particularly preferred.

Within the context of the present invention, the preferred glucan-containing matrices employed are fermentation broths, culture media and suspensions, as well as mycelia, hydrocolloids or powder preparations, which have a solvent proportion of from 20 to 99.9% by weight, and, in particular, of from 50 to 99% by weight, in each case based on the solid content. For example, the water-insoluble and glucan-containing mycelia in the form of solid compositions can be treated with the enzyme complex, with these solid compositions appearing particularly advantageous since they can be converted in a one-step reaction.

The present invention envisages, for the process, the preferred use of fermentation broths or culture media which contain unsolved solids, cell constituents and/or cell fragments. However, mycelia, hydrocolloids or powder preparations which are used as aqueous solutions are also equally especially well suited.

The polysaccharide which is employed in accordance with the invention is usually a hydrophilic colloid which is obtained by fermentation in a customary nutrient medium using microorganisms. Such glucans, for example in the form of scleroglucans, and their preparations, can be used in the form of the fermentation broths or the culture medium, in connection with which they can, as described, contain unsolved solids and cell constituents or cell fragments.

The process according to the invention is usually carried out by adding the protein having enzymatic activity to a matrix, which contains the polysaccharide, which is in the form of an aqueous solution and which also contains the insoluble constituents, and then leaving this mixture to stand, with it being of no significance whether this solution is stirred or not. A crucial criterion for the duration and success of the reaction is the time required for the enzymatically determined release of the mycelium-bound polysaccharides into the solution. Normally, it is entirely adequate for the aqueous solution to contain from 0.03 to 3.0% by weight of the polysaccharide. The concentration of the protein having enzyme activity which is employed always depends directly on the glucan concentration and on the quantity of the insoluble cell constituents which are contained therein.

As already indicated, the matrices which are used in the form of a mycelium, of a hydrocolloid or of a powder preparation can also contain certain proportions of solvents or be employed as aqueous solutions, with water being particularly preferably, according to the invention, used as the solvent for the matrix.

In order to achieve an optimal enzymatic conversion, a process is recommended in which the matrices employed are stirred in order, in this way, to prevent the solids from settling and to ensure that the concentration of the enzymatic activity-possessing proteins which are used is made uniform in the polysaccharide-containing solution.

While, taken overall, proteins having β(1,3)-glucanase activity and, especially, β(1,3)-glucanases have been found to be absolutely pH-tolerant, pH values which lie between 4.0 and 10.0 and, in particular, between 5.0 and 7.0 are recommended for the matrices in the case of the present process. The abovementioned conditions can ensure maximum release of the glucans from the insoluble cell material within relatively short periods of time. For this reason, the invention claims, for the duration of the enzymatic treatment, periods of time of between 15 minutes and 24 hours and, in particular, of between 1 to 6 hours.

While the claimed process can also be carried out batchwise, preference is given to a continuous process, with the protein possessing enzymatic activity being added to a recipient vessel containing the aqueous polysaccharides in the form of a diluted or undiluted fermentation broth or of an aqueous solution of the isolated glucan. Particularly in connection with continuous operation, it is recommended that the reaction vessel or the container be selected to be of adequate size, and the rate of addition of the enzyme and the polysaccharide be stipulated, such that sufficient time is available to the aqueous polysaccharide solution containing the solid cell constituents, in the presence of an adequate concentration of enzyme, for the desired cell degradation and for the release of the polysaccharides.

The present invention also encompasses a process variant in which, after it has been treated enzymatically, the glucan-containing matrix is subjected to a heat treatment at temperatures of between 70 and 150° C. and, in particular, of between 80 and 140° C. In this connection, the heat treatment should be carried out for from 1 to 60 minutes and, in particular, for from 2 to 30 minutes. This heat treatment serves, in particular, to inactivate microorganisms and/or enzymatically active proteins.

In conclusion, the glucan-containing matrices can be subjected to a filtration and/or centrifugation, with this also being envisaged by the present invention. The filtration process can, for example, be carried out using a filter press and, where appropriate, using a filtration aid with, in any case, a purified glucan being obtained as the product.

For the purpose of completing the claimed process, the glucan can, in accordance with the present invention, be separated off from the enzyme-treated and, where appropriate, filtered glucan-containing solution, which separation should be effected, in particular, by means of evaporation, freeze-drying or precipitation. In the case of evaporation, the water is removed by heating; the glucan can be precipitated by adding alcohols while solvent (residues) can be removed by filtration. For successfully evaporating the water by means of heat, a temperature range of between 80 and 100° C. is proposed; a temperature of 20° C. and a pressure of 0.01 hPa are proposed for the freeze drying. If a precipitation step was to be carried out, the polysaccharide-containing solution is then added to pure alcohol. The precipitate is subsequently removed by filtration using a filter sieve and the solid which has been separated off is dried at room temperature (approx. 25° C.).

In addition to the process for preparing a β-1,3-glucan, the present invention also claims a β-1,3-glucan which is prepared using this process and, in particular, a corresponding glucan which possesses improved solubility in cold water and/or reduced proportions of insoluble constituents and/or an increased viscosity and/or a reduced turbidity in aqueous solutions and/or improved filterability.

However, the present invention also relates to a solid formulation which comprises at least from 90 to 99.9% by weight of an untreated β-1,3-glucan and also from 0.005 to 0.1% by weight of a protein having β(1,3)-glucanase activity and also from 0 to 10% by weight of at least one additional ingredient, such as fillers, inert diluents or a mycelium. In this connection, the β-1,3-glucan employed can in turn possess β(1,6)-glucose side chains and the protein which is used can additionally exhibit β(1,4)-glucanase activity. These formulations can be introduced directly, in solid form, into water or other aqueous media, with the formulations possessing the advantage that enzymes and polysaccharides do not have to be added separately.

Finally, the present invention also claims the use of a β-1,3-glucan, which has been obtained using the described preparation process, for cosmetic applications and/or in body care and health care and/or in the food industry and/or in oil production.

In summary, it can be stated that the present invention makes available an improved process for preparing β-1,3-glucans, with the yields being markedly higher and the quality of the glucans obtained by this process, and in particular of the scleroglucans, being markedly improved by enzymatic treatment of the crude fermentation broths or of the glucan powder. In addition, it is possible to use this process, by means of an enzymatic treatment, in order to liquefy insoluble mycelium constituents, by releasing mycelium-bound polysaccharides, resulting in the polysaccharide-containing solutions having a higher viscosity.

The following examples clarify the abovementioned advantages of the claimed process for preparing β-1,3-glucan and thus of the resulting glucans.

### EXAMPLES

### Example 1

### Increasing the Viscosity of a Scleroglucan-Containing Solution

1 g of scleroglucan (Actigum CS6, Degussa AG) was added to 100 ml of distilled water and the mixture was stirred at 20° C. for 24 hours using a propeller agitator. 10 ml of this scleroglucan-containing solution were then added, at 37° C. and using a shearing rate of 10/second, to a Thermo Haake viscometer (Rotovisco C1). 1 ml of a solution containing, as the enzyme, 1.53 mg of an endo-β(1,3)-glucanase (Megazyme)/ml of distilled water was then added and the measurement was begun.

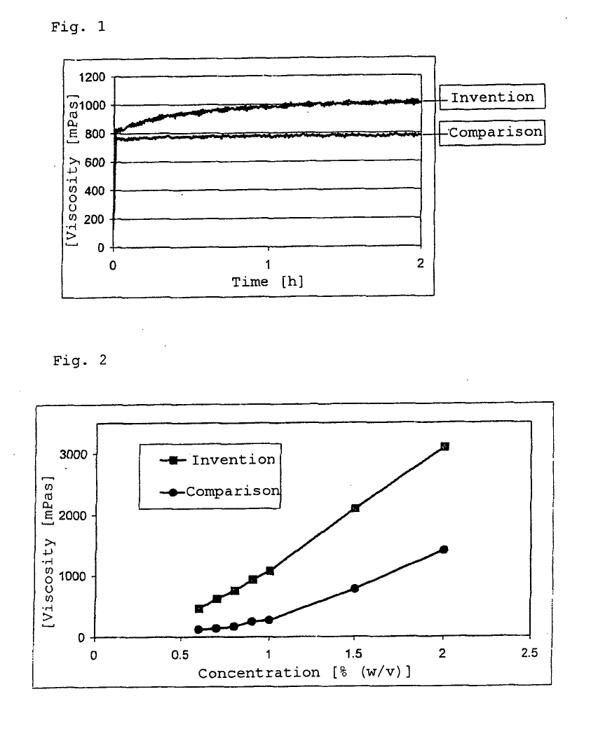

The results of this example are depicted in FIG. 1. As compared with the reference sample, the viscosity of the enzyme-treated sample increased by approx. 30% within 2 hours.

### Example 2

### Enzymatically Modifying Scleroglucan

1 g of scleroglucan (Actigum CS6, Degussa AG) was added to 100 ml of distilled water and the mixture was stirred at 20° C. for 24 hours using a propeller agitator. The solution was then warmed to 37° C. and 1 ml of an enzyme solution containing 2 mg of 1,3-β-glucanase (Glucanex, Novozymes)/ml of distilled water was added. This solution was kept at 37° C. while stirring constantly for 3 hours and then added to 1000 ml of pure alcohol (VWR No. 100943). The insoluble precipitate was then separated off from the solution using a filter sieve (mesh width 70 μm) and the precipitate which had been separated off was dried at 20° C. and pulverized using a mill.

A sample which had been prepared in a corresponding manner but in which 1 ml of distilled water had been added instead of the enzyme solution, serves as the comparison. The viscosity of the enzyme-treated sample according to the invention and of the reference sample were determined, at 20° C. and at a shearing rate of 10/second, using a Thermo Haake viscometer (Rotovisco C1).

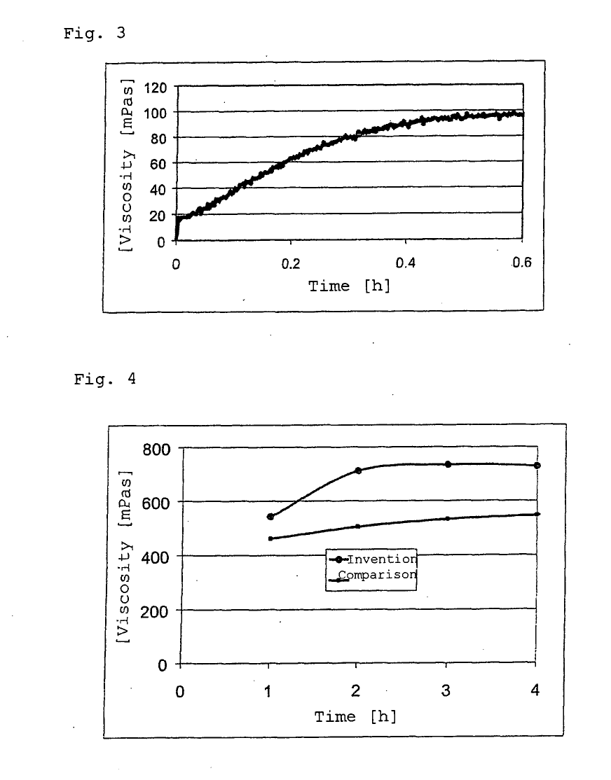

FIG. 2 shows that the viscosity of the scleroglucan which was modified in accordance with the invention is almost twice as high as that of the reference sample which was treated under comparable conditions.

### Example 3

### Enzymatically Treating an Insoluble Mycelium

1 g of scleroglucan (Actigum CS6, Degussa AG) was added to 100 ml of distilled water and the mixture was stirred at 20° C. for 24 hours using a propeller agitator. The mixture was then centrifuged at 1500 rcf for 30 minutes, after which the solution was removed and 500 ml of distilled water were added to the sediment. The resulting suspension was stirred with a magnetic stirrer for 30 minutes and the previously mentioned steps (centrifuging, removing the supernatant, taking up once again and stirring the suspension) were repeated five times. After the last centrifugation step, the solution was removed and the residue was frozen at −20° C. The frozen residue was then freeze-dried at 0.01 hPa for 24 hours after which 50 mg of the residue were suspended in 10 ml of distilled water. 1 ml of an enzyme solution containing 2 mg of 1,3-β-glucanase (Glucanex, Novozymes)/ml of distilled water were added to this suspension and the resulting solution was added, at 37° C. and at a shearing rate of 10/second, to a Thermo Haake viscometer (Rotovisco C1). The measurement was then started.

FIG. 3 shows that the viscosity of the mycelium suspension which was enzymatic treated in accordance with the invention increased by more than ten-fold, suggesting that the polysaccharide was released from the insoluble mycelium, and dissolved in the water, during the enzymatic treatment. At the same time, a decrease in insoluble mycelium particles was observed.

### Example 4

### Enzymatically Treating a Sclerotium Fermentation Broth

520 g of a scleroglucan broth (Degussa AG) were added to 2080 g of distilled water and the mixture was stirred at 25° C. for 2 hours using a high-shearing agitator; the pH of the solution was then adjusted with a 10% solution of NaOH to values of between 5.2 and 5.4 after which 2.6 g of an enzyme powder (Safizym CP, Saf-isis) were added to the solution, which was then divided into portions of 200 g. The individual portions were then placed, at 37° C., in an orbital shaker for periods of between 1 and 6 hours. After in each case one hour, individual portions were removed from the shaker, the solutions were separated off and the viscosity was determined using a Brookfield rheometer (LVTD, 30 rpm).

FIG. 4 shows the result of the enzymatic treatment, according to the invention, of the fermentation broth:

Within 2 to 4 hours of the period of treatment, the polysaccharide was released from the insoluble mycelium and dissolved; the viscosity of the solution increased and reached a value which was by 40% higher than that of the reference samples which were prepared under comparable conditions but without any addition of enzyme.

### Example 5

### Preparing a Purified Scleroglucan

150 g of scleroglucan (Actigum CS6, Degussa AG) were dissolved in 20 l of distilled water while stirring with a high-performance agitator at 80° C. for 2 hours.

The solution was then cooled down to 37° C. and 24 g of Safizym CP (Saf-isis) were added. This suspension was stirred at 37° C. for 2.5 hours after which the solution was heated once again to 80° C. 500 g of filtering earth (FloM, CECA) were then added and the suspension was filtered using a filter press (Eurofiltec). The filtrate was added to 40 liters of 80% ethanol and the resulting coagulate was filtered using a filtering sieve (mesh width 70 μm). Finally, the coagulate was dried at 60° C. in an oven and ground using a grid grinder (Retsch).

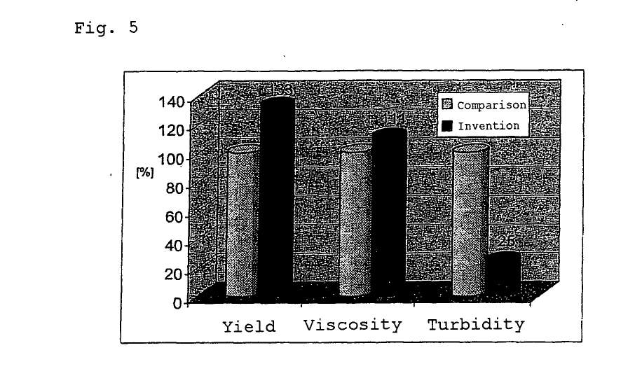

A reference sample which was prepared under the identical conditions, but without any addition of enzyme, served as the comparison. Comparing this reference sample and the scleroglucan sample which was enzyme-treated in accordance with the invention shows (FIG. 5) that, when using the process according to the invention, the yield after the filtration step was approx. 30% higher than that for the reference sample, with the viscosity of the filtered scleroglucan also being 10% higher. Finally, the turbidity of the sample according to the invention was lower than that of the reference sample by a factor of 4.

1-22. (canceled)

23. A process for preparing a β-1,3-glucan, comprising stirring a glucan-containing matrix at temperatures of between 15 and 60° C. and at a pH of between 4.0 and 10.0, in water as solvent and then treating with a protein possessing β(1,3)-glucanase activity.

24. The process as claimed in claim 23, wherein the β-1,3-glucan has β(1,6)-glucose side chains.

25. The process as claimed in claim 23, wherein the protein is β(1,3)-glucanase.

26. The process as claimed in claim 23, wherein the protein has β(1,4)-glucanase activity.

27. The process as claimed in claim 23, wherein the concentration of the protein having β(1,3)-glucanase activity is from 0.001 to 3.0% by weight based on the reaction mixture.

28. The process as claimed in claim 23, wherein the process is carried out at temperatures of between 20 and 40° C.

29. The process as claimed in claim 23, carried out at room temperature.

30. The process as claimed in claim 23, wherein the glucan-containing matrix is a fermentation broth, a culture medium, a suspension or a mycelium, a hydrocolloid or a powder preparation containing a solvent proportion of from 20 to 99.9% by weight and, in particular, of from 50 to 99% by weight based on the solids content.

31. The process as claimed in claim 23, wherein the fermentation broth or the culture medium contains unsolved solids, cell constituents and/or cell fragments.

32. The process as claimed in claim 23, wherein the mycelium, the hydrocolloid or the powder preparation is employed as an aqueous solution.

33. The process as claimed in claim 23, wherein the matrix contains water as solvent.

34. The process as claimed in claim 23, wherein the pH of the matrices is between 5.0 and 7.0.

35. The process as claimed in claim 23, wherein the duration of the enzymatic treatment is from 15 minutes to 24 hours and, in particular, from 1 to 6 hours.

36. The process as claimed in claim 23, wherein it is carried out continuously.

37. The process as claimed in claim 23, wherein after the glucan-containing matrix has been treated enzymatically, it is subjected to a heat treatment at temperatures of between 70 and 150° C.

38. The process as claimed in claim 37, wherein the heat treatment is carried out for 1 to 60 minutes.

39. The process as claimed in claim 23, wherein the glucan-containing matrices are finally subjected to a filtration and/or centrifugation.

40. The process as claimed in claim 23, wherein the glucan is separated off.

41. The process as claimed in claim 40, wherein separation is by evaporation freeze-drying or precipitation.

42. A β-1,3-glucan obtained by the process of claim 23.

43. A β-1,3-glucan as claimed in claim 42, wherein that exhibits improved solubility in cold water and/or reduced proportions of insoluble constituents and/or an increased viscosity and/or reduced turbidity in aqueous solutions and/or improved filterability.

44. A solid formulation comprising at least from 90 to 99.9% by weight of an untreated β-1,3-glucan, from 0.005 to 0.1% by weight of a protein having β(1,3)-glucanase activity and from 0 to 10% by weight of at least one further constituent, such as fillers, inert diluents or a mycelium.

45. A cosmetic comprising the β-1,3-glucan of claim 42.

46. A food product comprising the β-1,3-glucan of claim 42
